(19) United States
(12) Patent Application Publication
(10) Pub. No.: US 2008/0039849 A1
(43) Pub. Date: Feb. 14, 2008

(54) Application Aid for the Treatment of Bone Defects

(76) Inventor: Arne Briest, Karlsruhe (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/594,272

(22) PCT Filed: Mar. 24, 2005
(86) PCT No.: PCT/EP05/03141

§ 371 Date: Jul. 9, 2007

(30) Foreign Application Priority Data
Mar. 26, 2004 (DE) 10 2004 016 397.9

(51) Int. Cl. A61B 17/58

(52) U.S. Cl. 606/86

(57) ABSTRACT

Disclosed is an application aid (1) for introducing a material (12) used for the treatment of bone defects into a natural bone or a natural bone structure. Said application aid (1) comprises a largely flexible, tubular or sleeve-shaped outer part (2) that is composed of at least two components (3, 4) which can be coaxially moved into each other, a largely flexible, preferably tubular or sleeve-shaped inner part (7), and a discharge port (11) for the material, preferably on the outer part (2). The inventive application aid is preferably embodied such that at least one element which can be moved radially outward at least in part is provided in the interior. Said element represents particularly a balloon.

The invention relates to an application aid for introducing a material for treatment of bone defects into a natural bone or a natural bone structure, and to a kit that comprises such an application aid.

To treat bone defects effectively, a very wide variety of inorganic, organic or biological materials are used. These materials can serve to fill the bone defects, but can also promote regeneration of the bone, for example by means of suitable growth factors.

However, the application of the corresponding material for the treatment of bone defects often proves difficult. Whereas in certain bone structures, for example the jaw bone, it is fairly simple to introduce the material into the defective bone, it is difficult by comparison in other bone structures, for example in tubular bones and in the vertebrae. This is true especially of materials that cannot be applied in a liquid form or in a form with relatively low viscosity, but are instead present in the form of a solid.

A large number of application aids are known in the medical sector, but they are principally intended for the treatment of blood vessels and for vascular surgery. However, such application aids, for example what are known as stents, are unsuitable for the treatment of bone defects.

The object of the invention is, accordingly, to make available an application aid with which material for treatment of bone defects can be introduced into a natural bone or into a natural bone structure. This application aid should in particular also be able to deliver materials in the form of solids, for example with a cotton wool consistency, into bone structures that are relatively difficult to access, such as tubular bones or vertebrae.

This object is achieved by the application aid having the features of claim 1. Preferred embodiments of this application aid are set out in dependent claims 2 through 18. Claim 19 defines a kit that is used for treatment of bone defects and that comprises an application aid according to the invention. The wording of all the claims is herewith incorporated by reference into the content of this description.

According to the invention, the application for introducing the material for treatment of bone defects comprises
a substantially flexible and tubular or sleeve-shaped outer part which is composed of at least two components that are coaxially displaceable in one another,
a substantially flexible, preferably tubular or sleeve-shaped inner part, and
a discharge orifice for the material, preferably provided on the outer part.

The application aid according to the invention can also be described in other terms, that the outer part is composed of a kind of outer catheter and a kind of inner catheter. By means of the coaxial displacement of the outer catheter relative to the inner catheter, the material contained in the application aid is discharged from the application aid through the discharge orifice. Inside the outer part composed of the outer catheter and the inner catheter, there is an inner part in the form of a further catheter, which is provided, for example, for the passage of a guide wire for the application aid. The function of all the parts of the application aid will be explained in more detail below in connection with the preferred embodiments and with reference to the figure.

The application aid according to the invention has gripping means which are preferably provided on the components of the outer part and which are used for displacing these components relative to one another/in one another. These gripping means are preferably handgrips, for example in the form of suitable protuberances, that allow the user to maneuver the application aid in the intended manner.

As has already been indicated, the application aid according to the invention preferably has at least one guide means with which the application aid is guided to the application site. Such guide means are known for example in the form of so-called guide wires. The guide means extends substantially along the entire length of the application aid. It is preferably arranged inside the inner part, which can preferably be tubular or sleeve-shaped in the form of a catheter.

In a development of the invention, the discharge orifice for the material is provided at that end of the application aid later directed toward the application site, this end preferably being designed in the shape of a tip. This makes it easier to advance the application aid in the direction toward the application site. In preferred embodiments, this tip with the discharge orifice is located on the outer part. In such embodiments, however, the application aid can also be designed such that the tip with the discharge orifice is located on the inner part. This can be done, for example, by means of a catheter, provided as an inner part and with a comparatively small cross section, first widening at the corresponding end and then narrowing again toward the discharge opening in order to allow the material to emerge through the discharge opening. Correspondingly, provision can also be made for a corresponding component, that forms the tip with discharge opening, to be fitted onto the inner part.

To permit easy discharge of the material from the application aid, that end of the application aid directed toward the application site and comprising the discharge orifice can be constructed such that the discharge orifice widens as the material is discharged. This can be achieved, for example, by the corresponding end of the application aid being made from a thinner material than the rest of the body of the application aid. If appropriate, it is also possible for another material to be used for the end than for the rest of the body of the application aid, which material can then be more easily extensible so that the discharge orifice widens as the material is discharged. These embodiments are particularly advantageous when, as has already been described, the discharge orifice is located on an end of the application aid shaped in the manner of a tip.

In other preferred embodiments of the application aid according to the invention, at least one element that can at least partially be moved radially outward is provided in the inside of the outer part. This element preferably has a membrane-like or film-like design. Depending on the intended use, the element, preferably the membrane or film, can be impermeable or permeable, for example porous. Permeable membranes/films permit, for example, an exchange of substances through the membrane/film and also growth of bone through the membrane/film. Moreover, the element, preferably the membrane or film, can have a sufficient mechanical strength and stiffness to provide a support function for the applied material and, if appropriate, the growing bone.

In the embodiments with the additional element, the effect achieved is that the material contained in the application aid can be moved or pressed radially outward before and in particular after the discharge of the material from the application aid, in order to introduce said material into the bone or the bone structure. The element provided in the inside of the outer part thus assumes, at least for a short time, a support function for the material, in order to convey it into the bone structure and keep it there. This is especially advantageous in tubular bones or vertebral bones when the material provided for treatment of bone defects is to be deposited in the interior of the tubular bone or vertebra, in particular to be placed on the inside walls of these bone structures. This can be done by moving the element or elements radially outward, for example by tensioning or pressurizing these elements.

In the last-described preferred embodiments of the application aid according to the invention, said elements are preferably secured on the inner part or at least partially form the inner part themselves. This has the advantage of a simplified construction.

To move the elements radially outward, the elements can preferably be arranged on the outer surface of a balloon-like element. In an alternative preferred embodiment, the elements themselves form the wall of a balloon-like element. In both cases, the balloon-like element ensures that the material for the treatment of bone defects can be moved or pressed radially outward. In line with the above embodiments, the balloon-like element is preferably secured on the inner part or forms part of the inner part. Such embodiments are configured, for example, such that the balloon-like element is secured at at least two places on the outer circumference of the inner part and, in this way, the volume of the balloon between the inner part and the inside wall of the balloon-like element is defined. Such an embodiment is also represented in the drawing appended to this application document. In other such embodiments, the balloon-like element forms part of the inner part itself. The inner part is in this case closed at the end directed toward the application site and widens toward the balloon-like element. In this way, the interior of the balloon-like element is accessible from the outside via the inner part and can thus be filled, for example, with the material for the treatment of bone defects.

In the described embodiments with a balloon-like element, this balloon-like element is preferably inflatable. For this purpose, pressure, in particular air pressure, can be applied to the balloon-like element via the inner part itself or via a tube guided through the inner part. In the embodiments in which the balloon-like element forms an (integrated) part of the inner part, the balloon is inflated directly, i.e. again via the inner part. This is achieved by the fact that the part of the inner part leading to the balloon-like element is not inflatable, for example is made of metal, and is adjoined by the inflatable balloon-like element. In all the embodiments discussed, the pressure application can be reversible, i.e. after the balloon-like element has performed its function, the pressure can be removed, and the balloon-like element collapses in on itself again.

In alternative embodiments, the balloon-like element can preferably be self-expandable, i.e. it moves radially outward by itself, without pressure being applied to it. This can be achieved, for example, by the fact that the balloon-like element is introduced under external pressure into the application aid or by the fact that the balloon-like element is made of what is known as a shape-memory material. Such materials, for example metal alloys like NiTi, but also plastic materials in particular, are known to a person skilled in the art. Under certain conditions, for example temperature conditions, they recover their original shape and, as it were, thus remember this original shape. This shape-memory effect can be utilized in conjunction with the present invention. If, in these cases, the actual function of the balloon-like element is also intended to end after a certain period of time, the balloon-like element can be made to collapse by application of an external underpressure (suction pressure).

Depending on the intended medical use, the preferably membrane-like or film-like elements or the balloon-like element may or may not remain in the bone structure. If said elements or the balloon are intended to be removed again, this can be done most easily if it is secured on the inner part or at least partially forms the inner part. Removal is thus automatically guaranteed upon the removal/withdrawal of the application aid from the bone structure.

If said elements or the balloon-like element are intended to perform their function, in particular a support function, for a certain period of time after removal of the application aid, they are secured releasably, particularly on the inner part. In this way, all these elements can remain in the bone structure. The material from which the elements are made can in this case be chosen as a function of the respective medical situation. If the element, in particular the balloon, is intended to lose its function after a certain period of time, a bioabsorbable substance is preferably used. In principle, this can include metals such as magnesium or iron, which are also counted as being bioabsorbable. However, the use of bioabsorbable plastics is preferred. Using bioabsorbable substances ensures that the element/balloon disintegrates after a certain time. If the element/balloon is intended to perform its function for an indefinite time, a biocompatible but non-bioabsorbable material is used, for example a biocompatible metal such as titanium. In this way, the construction of the application aid according to the invention can be adapted to a very wide variety of possible uses.

Depending on the medical situation, the structure or surface of the elements used, including the balloon, can also be modified. For example, in order to achieve good regeneration of bone, the element or the balloon can be made from a porous membrane-like material, so that the bone is able to grow through this membrane and an exchange of substances can take place through the membrane. Cases are also conceivable, however, in which such a membrane-like structure of the element is not of any advantage, so that substantially closed films can be used in such cases.

In a further development, marking means, in particular for X-ray detection, can be provided on the outer part and/or on the inner part of the application aid according to the invention. In this way, it is possible, for example, to monitor the displacement of the components of the outer part in one another/relative to one another or the discharge of the material from the application aid.

In particularly preferred embodiments of the invention, the described application aid is filled with a material for the treatment of bone defects. This material can, in principle, include all inorganic, organic or biological materials that are known for such treatment of bone defects. Solid materials, particularly those with a relatively soft consistency, are preferably used for the filling. For example, these can be biological materials such as the ones sold by the Applicant under the names Colloss® and Targobone®.

In a further development of the filled application aid according to the invention, the outer part, i.e. the space defined inside the outer part, is filled at least partially, preferably completely, with the material.

In these embodiments it is preferred, on the one hand, if basically only the volume between the inner part or the optionally present preferably membrane-like or film-like elements (balloon) and the wall of the outer part is filled with the material. The material is thus situated between the outer surfaces of these elements or of the balloon and the (inside) wall of the outer part or its components.

On the other hand, it is also preferred if basically only the volume within the optionally present preferably membrane-like or film-like elements (balloon) is filled with the material. In these cases the material is thus situated only inside the space defined by the elements or inside the balloon.

However, preference is also given to an embodiment in which both the volume between the elements/balloon and the wall of the outer part and also the volume within the elements/balloon is filled.

Finally, as has been mentioned, the invention includes a kit for the treatment of bone defects, which kit comprises at least one application aid as described above.

As will be evident from the above description, the invention affords a whole series of advantages. With the claimed application aid, it is easily possible for the material provided for treatment of bone defects to be delivered directly to the application site in the bone structure. This applies in particular to bone structures that can be accessed only with difficulty by the physician carrying out the treatment. These include tubular bones or vertebrae, in particular. With the application aid, it is possible to discharge the preferably solid material and, in the embodiments with the movable elements or the balloon, to move or press this material against the defective bone structure. The material is thus delivered specifically to those locations where bone growth or regeneration of bone is intended to take place. Depending on the material used, the application aid can then provide a temporary or permanent support function for this material. It will often suffice if the material is pressed against the bone structure only for a comparatively short time. Many materials, in particular biological materials with a protein content, adhere by themselves to the bone structure, so that a permanent support is not required. In these cases, the supporting elements or balloon can be withdrawn again or can be made of a bioabsorbable material. If a longer-lasting support function is required, as is necessary for example for regeneration of vertebrae preceded by required straightening of the spine, these elements or the balloon can be made of a biocompatible material of sufficient strength that maintains its support function permanently or at least until such time that the growing bone itself can assume this support function.

The features described, and further features of the invention, will become clear from the attached drawing in conjunction with the claims. The individual features can be provided singly or in combination with one another.

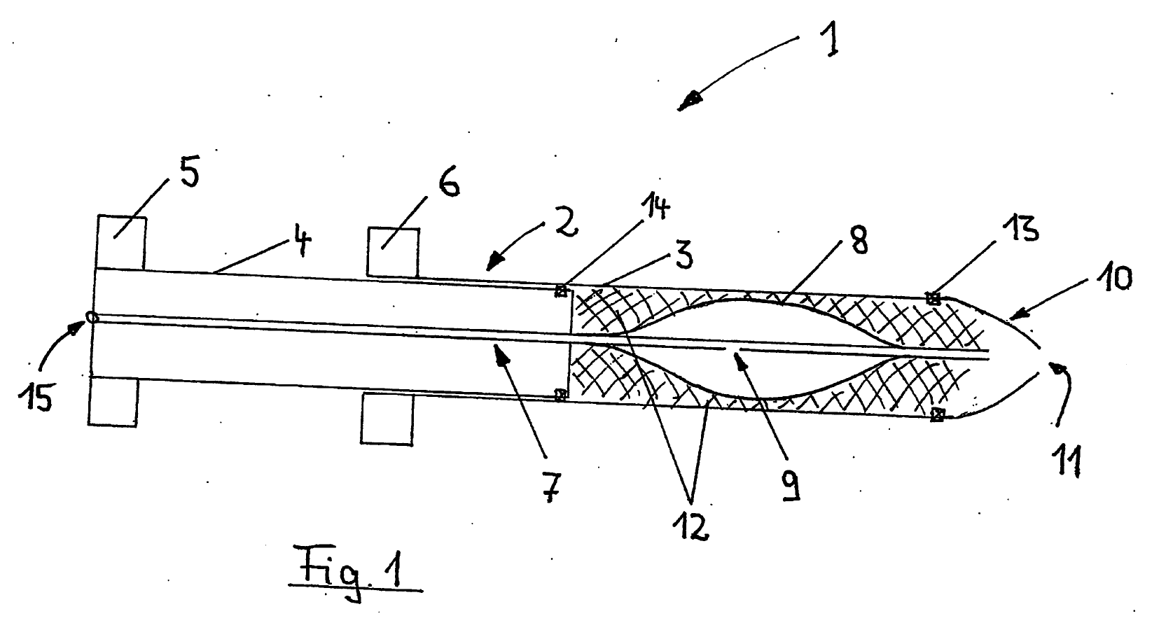

In the drawing, FIG. 1 shows a schematic cross-sectional view of an application aid according to the invention or applicator according to the invention.

The application aid 1 shown in FIG. 1 has an elongate tubular outer part 2 in the manner of a catheter, which is composed of the two components 3 and 4 that are fitted at least partially in one another. Accordingly, the components 3 and 4 can also be defined as outer catheter and inner catheter, respectively. Holding means or handgrips 5 and 6, with the aid of which the component 3 can be displaced relative to the component 4, are located on the outer circumference of the components 3 and 4 of the outer part 2.

In the inside of the outer part 2 there is a likewise elongate and tubular inner part 7 which is designed in the manner of a catheter and can accordingly also be designated as a (further) inner catheter. The inner part 7 and the components 3 and 4 of the outer part 2 are preferably made of plastic.

A guide wire (not shown) can be inserted inside the inner part 7, and the application aid 1 can be moved in the direction toward the application site in the body with the aid of this guide wire.

In the area of the application aid 1 directed toward the application site during its use (“forward”/distal; right-hand side in the figure), a (rotationally symmetrical) balloon-like element 8 is fitted on the inner part 7. Pressure can be applied from outside to this element 8 by way of the hole 9 provided in the inner part 7, for example via the (“rear”/proximal) adapter 15, and said element 8 can thus be radially expanded outward. In this way, the material 12 contained in the application aid 1 (between the balloon-like element 8 or inner part 7 and the wall of the component 3 of the outer part 2) for treatment of bone defects can also be moved radially outward and, for example, pressed against a bone structure into which the application aid 1 is advanced.

For discharging the material 12 from the application aid 1, the latter has a discharge orifice 11 at its end directed toward the application site (see right-hand side of FIG. 1), said discharge orifice 11 being provided on the end shaped as a tip 10 according to FIG. 1. To ensure that the orifice 11 widens during discharge of the material 12, the wall of the component 3 of the outer part 2 is thinner in the area of the tip 10 than in the other areas of said component 3 (this is not shown in the figure).

To be able to monitor the displacement of the component 3 of the outer part 2 relative to the component 4 of the outer part 2 by X-ray, suitable markings 13 and 14 are located on the outer circumference of the components 3 and 4, respectively.

An application aid 1 as shown in FIG. 1, contained for example in a kit, functions in the following way:

The application aid 1 filled with the material 12 is introduced by the physician into the patient's body in the normal way during the operation and is advanced to the intended application site. In the case of a tubular bone, a hole can be drilled for this purpose into the tubular bone, and the application aid 1 can be inserted under X-ray control into the interior of this tubular bone. When the application site is reached, the component 3 of the outer part 2 is moved back relative to the component 4 of the outer part 2 until the balloon-like element 8, with the material 12 surrounding it, protrudes at least partially from the discharge orifice 11. The balloon-like element 8 (which if appropriate may already be pressurized) is then pumped up further and expanded in order to deliver the material 12 into the interior of the bone structure, for example to press it against the inside wall of the tubular bone. In the case where the balloon-like element 8 remains in the bone, said element 8 is then uncoupled from the inner part 7 (this is not shown in FIG. 1), or the balloon is allowed to collapse and is withdrawn from the bone again together with the application aid 1.

1. An application aid for introducing a material for treatment of bone defects into a natural bone or a natural bone structure, comprising
a substantially flexible and tubular or sleeve-shaped outer part which is composed of at least two components that are coaxially displaceable in one another,
a substantially flexible, preferably tubular or sleeve shaped inner part, and
a discharge orifice for the material, preferably on the outer part.

2. The application aid as claimed in claim 1, further comprising gripping means, provided on the components of the outer part for displacing them in one another.

3. The application aid as claimed in claim 1, further comprising at least one guide means extending substantially along the entire length of the application aid.

4. The application aid as claimed in claim 1, wherein the discharge orifice for the material is provided at an end of the outer part, designed in the shape of a tip.

5. The application aid as claimed in claim 1, wherein, in the inside of the outer part, at least one membrane-like or film-like element is provided that can be moved at least partially radially outward.

6. The application aid as claimed in claim 5, wherein the at least one membrane-like or film-like element is secured on the inner part or at least partially form the inner part.

7. The application aid as claimed in claim 5, wherein the at least one membrane-like or film-like element is arranged on the outside surface of a balloon-like element.

8. The application aid as claimed in claim 5, wherein the at least one membrane-like or film-like element forms the wall of a balloon-like element.

9. The application aid as claimed in claim 8, wherein the balloon-like element is inflatable, the inner part itself being able to be subjected to pressure and/or a tubular element being guided through the inner part.

10. The application aid as claimed in claim 8, wherein the balloon-like element is self-expandable.

11. The application aid as claimed in claim 6, wherein the at least one membrane-like or film-like element is secured releasably.

12. The application aid as claimed in claim 5, wherein the least one membrane-like or film-like element is made of a bioabsorbable material.

13. The application aid as claimed in claim 1, further comprising marking means for detecting the relative displacement of the components of the outer part and/or for detecting the discharging of material.

14. The application aid as claimed in claim 1, wherein the application aid is filled with a material for treatment of bone defects.

15. The application aid as claimed in claim 14, wherein the outer part is filled at least partially with the material.

16. The application aid as claimed in claim 15, wherein only the volume between the inner part and/or the at least one membrane-like or film-like element and the wall of the outer part is filled with the material.

17. The application aid as claimed in claim 15, wherein only the volume to the inside of the at least one membrane-like or film-like element is filled with the material.

18. The application aid as claimed in claim 15, both the volume between the at least one membrane or film-like element and the wall of the outer part and also the volume within these elements is filled with the material.

19. A kit for the treatment of bone defects, comprising at least one application aid as claimed in claim 1.

20. The application aid as claimed in claim 1, wherein the inner part is tubular or sleeve-shaped.

21. The application aid as claimed in claim 1, wherein the discharge orifice is provided on the outer part.

22. The application aid as claimed in claim 2, wherein the gripping means comprise handgrips.

23. The application aid as claimed in claim 3, wherein the guide means comprises a guide wire.

24. The application aid as claimed in claim 7, wherein the balloon-like element is inflatable, the inner part itself being able to be subjected to pressure and/or a tubular element being guided through the inner part.

25. The application aid as claimed in claim 7, wherein the balloon-like element is self-expandable.

26. The application aid as claimed in claim 6, wherein the at least one membrane-like or film-like element is made of a bioabsorbable plastic.

27. The application aid as claimed in claim 7, wherein the balloon-like element is made of a bioabsorbable material.

28. The application aid as claimed in claim 8, wherein the balloon-like element is made of a bioabsorbable plastic.

29. The application aid as claimed in claim 10, wherein the balloon-like element is made of a shape-memory material.

30. The application aid as claimed in claim 25, wherein the balloon-like element is made of a shape-memory material.

31. The application aid as claimed in claim 23, wherein the guide wire extends inside the inner part.
